(19) United States
(12) Patent Application Publication
(10) Pub. No.: US 2011/0137145 A1
(43) Pub. Date: Jun. 9, 2011

(54) METHOD FOR COMBINING HIGHLY PERMEABLE PARTS OF A MAGNETIC SHIELD

(76) Inventors: Sergio Nicola Erné, Neu-Ulm (DE); Hannes Nowak, Isserstedt (DE); Georg Bison, Jena (DE)

(21) Appl. No.: 12/966,667

(22) Filed: Dec. 13, 2010

Related U.S. Application Data
(63) Continuation of application No. PCT/EP2009/004260, filed on Jun. 12, 2009.

(30) Foreign Application Priority Data
Jun. 13, 2008 (DE) DE 102008028259.6

(51) Int. Cl. A61B 5/05; H01F 7/00; B23P 11/00

(52) U.S. Cl. 600/409; 335/301; 29/428

(57) ABSTRACT

An inventive magnetic filling material for bridging interspaces in magnetic shields for shielding static or low-frequency magnetic fields. The magnetic filling material comprises at least one matrix material and at least one magnetic component embedded into the matrix material. The magnetic component has magnetically shielding properties.

### RELATED APPLICATIONS

This application is a continuation of PCT/EP2009/004260, filed Jun. 12, 2009, which claims priority to DE 10 2008 028 259.6, filed Jun. 13, 2008, both of which are hereby incorporated by reference in their entirety.

### BACKGROUND

The invention relates to a magnetic filling material and a magnetic shield. Such filling materials and magnetic shields can be used, in particular, for biomagnetic measurement systems, in particular in the field of magnetocardiology or magnetoneurolgy. However, application in other fields of natural sciences, medicine and technology is also possible, in principle. Furthermore, the invention relates to a method for producing a magnetic shield, and to a method for producing a magnetic filling material.

In recent years and decades, magnetic measurement systems, which until then were substantially reserved for pure research, have made inroads into many areas of technology, natural sciences and medicine. Neurology and cardiology, in particular, benefit from such biomagnetic measurement systems. The present invention, notwithstanding the possibility of use in other fields of natural science and technology and without restricting such further possibilities of use, is described below substantially with regard to biomagnetic measurement systems.

Biomagnetic measurement systems are based on the fact that most cell activities in human or animal bodies are associated with electrical signals, in particular electric currents. The measurement of these electrical signals themselves that are brought about by the cell activity is known from the field of electrocardiography, for example. Besides the purely electrical signals, however, the electric currents are also associated with a corresponding magnetic field, the measurement of which is utilized by the various known biomagnetic measurement methods.

While the electrical signals and the measurement thereof outside the body are linked by various factors such as, for example, the different electrical conductivities of the tissue types between the source and the body surface, magnetic signals permeate these tissue regions virtually without any disturbance. The measurement of these magnetic fields and the changes thereof thus allows conclusions to be drawn about the currents flowing within the tissue, for example electric currents within the cardiac muscle. A measurement of these magnetic fields with high temporal and/or spatial resolution over a certain region thus permits imaging methods which, by way of example, can reproduce a present situation of different regions of a human heart. Other known applications lie in the field of neurology, for example.

However, the measurement of magnetic fields of biological samples or patients or the measurement of temporal changes in said magnetic fields, represents a major challenge from a metrological standpoint. Thus, by way of example, the magnetic field changes in the human body which are to be measured in magnetocardiography are approximately one million times weaker than the Earth's magnetic field. The detection of these changes therefore requires extremely sensitive magnetic sensors. In most cases, therefore, superconducting quantum interference devices (SQUIDs) are used in the field of biomagnetic measurements. Such sensors generally, in order to achieve or maintain the superconducting state, have to be cooled typically to 4° K (−269° C.), for which purpose liquid helium is usually used. The SQUIDs are therefore generally arranged individually or in a SQUID array in a cryostat vessel (a so-called Dewar vessel) and are correspondingly cooled there. Alternatively, laser-pumped magneto-optical sensors are currently being developed, which can have approximately comparable sensitivity. In this case, too, the sensors are generally arranged in an array arrangement in a container for temperature stabilization.

The measurement of the extremely weak magnetic fields or the changes thereof, which are in the picotesla or sub-picotesla range, is naturally extremely sensitive to electromagnetic and magnetic interference. The magnetic field detectors, of whatever type, have to be read, for which purpose a multiplicity of electronic devices are known. However, these read-out electronics react sensitively to extraneous electromagnetic fields coupled in, which can cause great interference. Further interference results from the strong signal background of external magnetic fields such as, in particular, micropulsations of the earth's magnetic field or of other magnetic fields, in particular magnetic fields that vary temporally, such as are brought about in diverse ways in industrial society (e.g. through movement of large ferromagnetic masses such as, for example, trains, trucks, etc.).

The prior art discloses various approaches for solving the problem of the interfering influences. Many of these approaches are based on corresponding shielding against electromagnetic and/or magnetic fields. Thus, eddy current shields against alternating electromagnetic fields have long been known from the civilian (e.g. medical) and military sectors, which shields can be configured both in stationary fashion and in movable fashion. Shields composed of soft-magnetic materials are generally used against low-frequency influences.

EP 0 359 864 B1 describes a device and a method for measuring weak, location- and time-dependent magnetic fields. The device comprises a mounting device for receiving the examination object and also a sensor arrangement comprising a SQUID array. A magnetic shielding chamber is furthermore described, which has a shielding factor of at least 10 for alternating magnetic fields having a frequency of 0.5 Hz, a shielding factor of at least 100 for alternating magnetic fields having a frequency of 5 Hz and a shielding factor of at least 1000 for alternating magnetic fields having a frequency of 50 Hz or higher. Moreover, the shielding chamber has shielding factors of at least 1000 for high-frequency alternating fields (frequencies greater than 10 kHz).

One disadvantage of the device described in EP 0 359 864 B1 is that despite the complex shield, there are numerous connections between the internal region of the shielding chamber and the external region, which are caused, for example, by the mount of the patient couch being led through the base shield and also by further numerous posts and electrical leadthroughs that are led through the shield. These leadthroughs have the effect, however, that magnetic and electromagnetic fields are coupled into the interior of the shielding chamber and can considerably impair the measurement there.

These disadvantages of EP 0 359 864 B1 reflect a general problem posed, in particular, in the case of large-volume shields of sensors and measuring devices against the influence of both static and low-frequency magnetic fields, that is to say fields up to 10 Hz. If shielding elements in the form of highly permeable materials, that is to say magnetic shields having permeabilities of at least 100, are used, then these magnetic shields are generally joined together from individual parts. At the joining locations and also at other openings, however, weak points of the magnetic shields occur, which have a high reluctance and therefore overall greatly reduce the quality of the shield.

In the case of smaller enclosed volumes of the shields, the shields are often produced from one piece, and the shielding capacity corresponds to the typical permeability values of the shielding material used. In the case of large enclosed volumes, especially for accessible shielded rooms such as are used for biomagnetic measurements, for example, the shields are assembled from parts, however. Limits are imposed on the size of the shielding parts by the complex thermal treatment. In the case of such shields assembled from different parts, the shielding capacity does not correspond to the typical permeability values of the material used, but rather is lower, such that reference is made to an effective total permeability. The reason for this behavior is, as described above, transition regions at the seam locations between the different parts, which generally cannot be produced in a fully sealed manner for reasons appertaining to production technology. In particular on account of deformations as a result of thermal treatment, air gaps generally form and bring about a high reluctance between the parts.

### SUMMARY

These teachings provide devices and methods for shielding magnetic fields which are also suitable as large-volume shields and which also have a low reluctance in openings or in the transition region of individual shielding elements, such that overall the shielding properties are improved.

One exemplary concept consists in providing a magnetic shield against static and low-frequency magnetic fields, that is to say fields having frequencies of at most 10 Hz, by means of magnetic shielding elements. These shielding elements can be connected to one another in a connecting region or can have other types of openings. In particular, highly permeable materials, that is to say materials having a permeability of at least 100, can be used as shields. In order to avoid high reluctances in the connecting region between the shielding elements or in other types of openings, a filling material comprising a matrix material is introduced, which ensures the dimensional stability, and a magnetic component. In this way, at least one or all openings can be magnetically closed.

In a first aspect of these teachings, therefore, a magnetic filling material is proposed which can be used, in particular, for the shielding of jointing or other connecting regions or openings in or between the shielding elements of magnetic shields. Generally, the magnetic filling material is suitable for bridging openings in magnetic shields for shielding static or low-frequency magnetic fields, that is to say magnetic fields having a frequency of up to 10 Hz. The magnetic filling material comprises at least one matrix material, that is to say a carrier material, which substantially determines the form and the mechanical properties of the magnetic fillings produced by means of the magnetic filling material in the interspaces. In addition, the magnetic filling material comprises at least one magnetic component which is embedded into the matrix material and which has magnetically shielding properties.

The matrix material can have, in particular, substantially non-magnetic or diamagnetic or paramagnetic properties. Furthermore, the matrix material can have at least one deformable state and at least one cured state, that is to say can be an at least partly curable matrix material. In this case, the matrix material is liquid or has at least partly plastic and/or elastic properties in the deformable state. By way of example, the matrix material can have a pasty form and/or a viscosity of at least 100 mPas, preferably at least 1000 mPas, in the deformable state. In the cured state, by contrast, the magnetic filling material is intended to be substantially dimensionally stable under the forces that usually occur during operation, in particular under the influence of its own weight force, and is preferably intended to have mechanically supporting properties. In other words, the matrix material is preferably intended to be a curable matrix material or to comprise such a curable matrix material, wherein this term is also intended to encompass the situation in which the cured matrix material also still has at least slightly plastic and/or elastic properties.

Thus, by way of example, the matrix material can comprise an adhesive, that is to say a material having adhesive properties. Both single-component and multi-component adhesives can be used for this purpose. Furthermore, it is generally possible to use resins, in particular epoxy resins, without or especially with adhesive properties, multi-component adhesives or at least one component of a multi-component adhesive.

If a curable matrix material is used, then the curing can be effected in various ways. By way of example, a photochemical curing and/or a thermal curing can be affected. The admixture of a curing agent can also be realized. In this case, by way of example, the matrix material can comprise the main component of the multi-component adhesive, whereas the curing agent is only added later, or vice versa. Alternatively, the curing agent can also be wholly or partly a constituent of the matrix material.

These viscous, curable matrix materials are in contrast to the ferrofluids, colloidal suspensions of nanoparticles such as iron or nickel, for example, which can likewise be used in principle. Such ferrofluids are at best superparamagnetic, that is to say do not have sufficient permeability, and generally do not have the required long-term stability nor can they be positioned in a targeted manner owing to their low viscosity, for example at seam locations between shielding elements.

The magnetic component has magnetically shielding properties. For this purpose, the magnetic component can comprise, in particular, at least one material which has a permeability of at least 10, preferably of at least 100, in particular at least 300, and particularly preferably of at least 1000. In particular, metallic materials can be used as the magnetic component. By way of example, iron, nickel, nickel-iron alloys or similar materials can be used. In particular, iron-nickel alloys comprising a nickel fraction of between 60% and 90%, particularly preferably between 75% and 80% can be used. Such materials are commercially available as so-called “Mu-metals” for example in plate, granular or powdered form and can be used according to the invention as the magnetic component, embedded in the matrix material.

It is advantageous if the magnetic component is present in powder form or in particle form, wherein this powder and/or the particles are embedded in the matrix material, for example by dispersing, mixing or the like. It is desirable if the magnetic component comprises particles having an average particle size of less than 500 micrometers, in particular of less than 400 micrometers, in particular of less than 90 micrometers, and particularly preferably of approximately 60 micrometers. The maximum particle sizes, too, should preferably be below 500 micrometers, particularly preferably below 100 micrometers.

The magnetic component can comprise, in particular, a mass fraction of 5 to 50% by weight, in particular 10 to 30% by weight, and particularly preferably approximately 15% by weight, of the magnetic filling material and/or a volume fraction of approximately 1-30% by volume of the magnetic filling material.

As described above, the magnetic filling material can be used, in particular, in the context of magnetic shielding for shielding static or low-frequency magnetic fields. The magnetic shield proposed comprises at least one shielding element having magnetically shielding properties, preferably in turn having permeabilities of at least 10, preferably at least 100 or even at least 300 and particularly preferably at least 1000. The shielding element has at least one opening, wherein, for reinforcing the magnetic shield, at least one magnetic filling material in accordance with one or more of the embodiments described above is introduced into the opening. If a plurality of openings is present, then the magnetic filling material can be introduced into one, a plurality or all of said openings. The opening can also be provided at the edge of the at least one shielding element or between two partial shielding elements that are jointly in turn joined together to form a shielding element.

It is particularly preferred, as described above, if the magnetic shield is composed of a plurality of shielding elements in a modular fashion, since large-volume shields can also be realized in this way. Accordingly, it is preferred if the magnetic shield has at least two shielding elements having magnetically shielding properties. The shielding elements are connected to one another in at least one connecting region. For reinforcing the magnetic shield, at least one magnetic filling material in accordance with one or more of the embodiments described above is introduced in the connecting region.

The magnetic filling material can also be introduced in other regions in which undesired openings occur in or between the shielding elements and/or within a shielding element, for example in the region of leadthroughs or the like. Generally, as an alternative or in addition to connecting regions, the at least one opening can comprise, in particular, a leadthrough, a joining location, a seam location, a butt joint or combinations of the stated openings and/or further types of openings. Consequently, the term opening should generally be interpreted broadly and encompasses any type of interruption of the magnetic shielding elements in which the magnetic shield is weakened.

The shielding elements can comprise at least one metallic material, for example once again a metallic material having a permeability of at least 10, preferably a permeability of at least 100 or even 300 and particularly preferably having a permeability of at least 1000. By way of example, once again iron, nickel, alloys of these elements and particularly preferably once again so-called mu-metals can be used for this purpose. The shielding elements can be configured substantially as plate-type elements, for example, such that the magnetic shield can be composed of individual plates of the shielding elements. However, additional shielding elements in the form of connecting parts such as brackets, corners or the like are also possible, of course. Overall, the magnetic shield can have substantially a parallelepipedal form, for example, wherein the magnetic filling material can be introduced, in particular, at the edges and/or corners of the parallelepiped. As described above, the shaped parts of the magnetic filling material become apparent, in particular, in the case of large-volume magnetic shields that cannot be produced from one piece, such that it is particularly preferred if the magnetic shield in the context of the present invention encloses an interior space of at least 1 m³. In particular, said interior space can be configured as an accessible interior space, that is to say for example as an interior space with at least one access door in which at least one person can be situated.

In addition to the magnetic filling material and the magnetic shield in one of the embodiments described above, a biomagnetic measurement system within the meaning of the above description of such biomagnetic measurement systems is furthermore proposed. In particular, the measurement system proposed can be used in magnetocardiology or in magnetoneurology but other fields of use are conceivable, for example in other areas of natural sciences, technology or medicine. The biomagnetic measurement system accordingly comprises at least one magnetic sensor system for detecting at least one magnetic field. By way of example, the magnetic sensor system can comprise a SQUID array and/or other types of biomagnetic measurement systems, for example magneto-optical sensors.

Furthermore, the biomagnetic measurement system proposed comprises at least one magnetic shield in accordance with one or more of the embodiments described above. In addition, the biomagnetic measurement system can comprise further components. By way of example, evaluation systems for the magnetic sensor system can be provided for example electronic evaluation systems and/or data processing systems. Energy sources, positioning devices for patients or the like can also be provided, which, like the magnetic sensor system as well, can be arranged, in particular, in an interior space of the magnetic shield, for example a magnetic patient chamber comprising the magnetic shield.

In addition to the biomagnetic measurement system, a method for producing a magnetic shield in accordance with one or more of the embodiments described above is furthermore proposed. In this case, firstly at least two shielding elements are mechanically connected to one another, that is to say connected in such a way that the magnetic shield as such is already substantially dimensionally stable. In this case, the at least one connecting region is formed, for example in the form of butt joints. The magnetic filling material is subsequently introduced into this connecting region. This introduction can be effected in a deformable state, in particular, and the magnetic filling material can subsequently be cured, for example by simple waiting, by thermal action, by action of light or by (and this can already be effected before the introduction of the magnetic filling material) introduction of an additional chemical curing agent.

The at least two shielding elements can be connected to one another, in particular, by force-locking and/or positively locking connections, in particular by plug connections. However, other types of connection are also possible, in principle, for example cohesive connections, in particular welding connections.

A method for producing a magnetic filling material in accordance with one or more of the embodiments described above is furthermore proposed. For this purpose, firstly the at least one matrix material is provided, which is preferably liquid and/or pasty, that is to say still deformable. The at least one magnetic component is then mixed into this at least one matrix material. As described, this can be effected in powder or particle form, for example. The mixing-in can be affected, for example, by stirring or other types of dispersion. Kneading-in is also conceivable.

### BRIEF DESCRIPTION OF DRAWINGS

The above-mentioned aspects of exemplary embodiments will become more apparent and will be better understood by reference to the following description of the embodiments taken in conjunction with the accompanying drawing, wherein:

FIGS. 1A and 1B are perspective views in schematic form that show a magnetic shield and a method for producing the magnetic shield;

FIG. 2 is a schematic view showing a biomagnetic measurement system; and

FIG. 3 is a view showing a method for producing a magnetic filling material.

### DETAILED DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of the present invention.

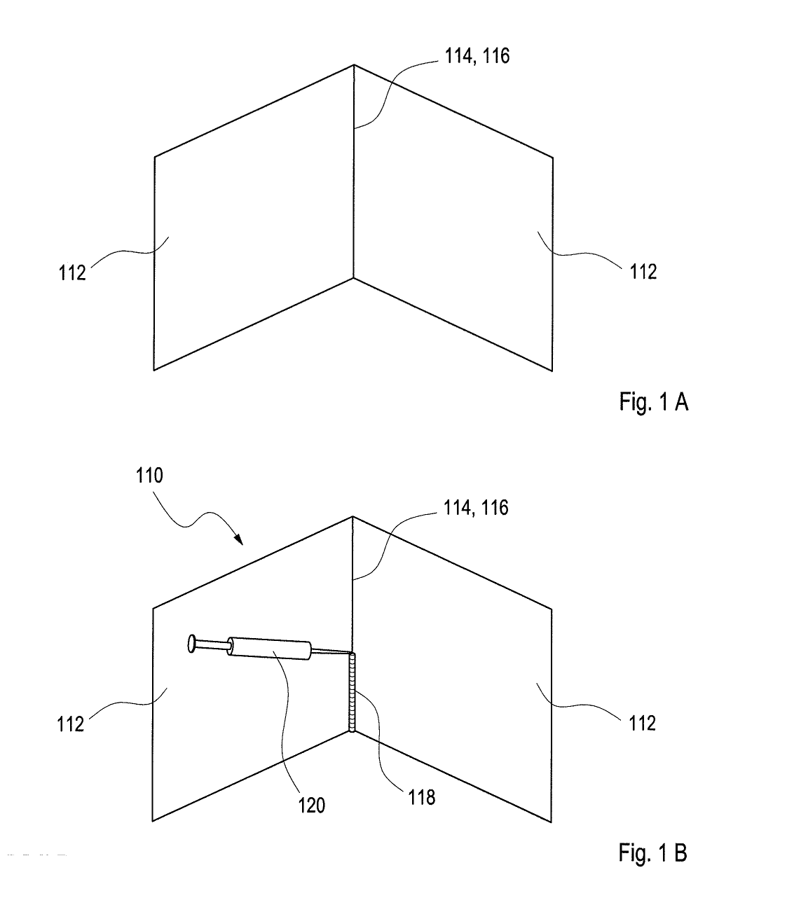

FIGS. 1A and 1B illustrate a method for producing a magnetic shield (designated by the reference numeral 110 in FIG. 1B) in a highly schematic form. In a first method step illustrated in FIG. 1B, two shielding elements 112 are joined together for this purpose. In the exemplary embodiment illustrated, the shielding elements 112 are embodied as plates, for example as plates of an iron-nickel alloy having a thickness of between 1 and 5 mm, for example. The shielding elements 112 can have a permeability of 300 or more, for example. The shielding elements 112 are mechanically connected to one another along a butt joint 114. By way of example, this mechanical connection can be effected by spot welding and/or by using connecting elements, such as, for example, clamps, connecting elements embodied in the shielding elements 112, or the like. By way of example, a plug connection can also be chosen. The butt joint 114 thus forms a connecting region 116 as an example of an opening in which the magnetic shield 110 is locally interrupted by the shielding elements 112.

In a second method step illustrated in FIG. 1B, this connecting region 116 is therefore sealed as completely as possible by a magnetic filling material 118. This magnetic filling material can be applied to the butt joint 114, for example, as illustrated in FIG. 1B, by means of a syringe 120 from the inner side and/or the outer side. Other types of introduction are also possible, for example application by blade, spraying, spreading or the like. For the purpose of introduction, the magnetic filling material 118 is initially preferably configured in a deformable fashion, for example as a paste. Details of the possible configuration of the magnetic filling material will be discussed more thoroughly below on the basis of the example in FIG. 3. In this way, by means of the method illustrated schematically in FIG. 1B, it is possible to produce a magnetically (tight) shield in which the connecting region or connecting regions 116 are also magnetically shielded by the magnetic filling material 118.

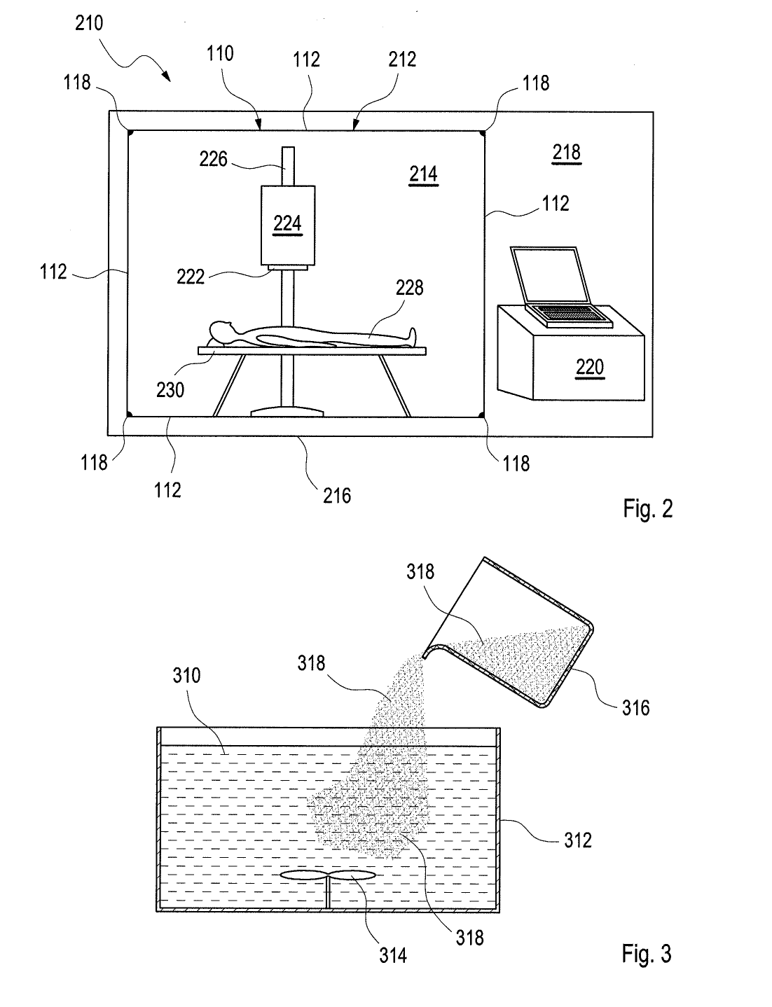

Since the magnetic shields 110 can then be constructed in an extremely modular fashion in accordance with the method according to the invention, without impairment by connecting regions, it is also possible to produce magnetic shields 110 having a large-volume interior space. Accordingly, FIG. 2 shows an exemplary embodiment of a biomagnetic measurement system 210 comprising a patient chamber 212 with a magnetic shield 110 according to the invention and an accessible interior space 214.

In the exemplary embodiment illustrated, the biomagnetic measurement system 210 comprises a measurement container 216, which has a shield against electromagnetic radio frequency fields, for example. In this measurement container 216, firstly an antechamber 218 is provided. This antechamber 218 can accommodate, for example, part of the driving and evaluation electronics 220 of the biomagnetic measurement system 210, for example an operator console. Further parts of the driving and evaluation electronics 220 can optionally be provided in the interior space 214 and/or outside the measurement container 216. The driving and evaluation electronics 220 can comprise, for example, one or a plurality of computer systems and also further electronic components.

Besides the antechamber 218, which can be accessible from outside through an external door (not illustrated in FIG. 2) for example, the patient chamber 212 is accommodated in the interior of the measurement container 216. Here as well, for example, a door connection can be provided between the antechamber 218 and the interior space 214, said door connection likewise not being illustrated in FIG. 2.

A magnetic sensor system 222 is provided in the interior space 214 of the patient chamber 212. This magnetic sensor system 222 can comprise a SQUID array, for example, which is mounted in a cooled fashion in a Dewar vessel 224, for example, and which is mounted in a height-adjustable fashion on a suspension device 226, for example.

By way of example, cardiac currents of a patient 228 lying on a patient couch 230 can be recorded by means of the magnetic sensor system 222. For shielding the magnetic sensor system 222 against static or low-frequency magnetic fields, the patient chamber 212 or the interior space 214 thereof is surrounded by the magnetic shield 110. In this case, this magnetic shield is configured in a parallelepipedal fashion, for example, and in turn comprises, for example, plate-type shielding elements 112 and also, analogously to FIG. 1B, in the connecting regions of said shielding elements 112 magnetic filling materials 118. Alternatively or additionally, other types of openings can also be sealed by said magnetic filling material 118, for example cable leadthroughs from the antechamber 218 into the interior space 214, for example for control lines or signal lines of the magnetic sensor system.

FIG. 3 symbolically illustrates a production method for producing a magnetic filling material 118. In this method, firstly a matrix material 310 is present in liquid and/or otherwise deformable form in a mixing vessel 312. The mixing vessel 312 has a stirrer 314, which is merely indicated symbolically in FIG. 3. Other types of dispersing devices are also possible, in principle; besides the stirrer 314, likewise optionally, by way of example, temperature-regulating devices or the like can also be present in order to lower the viscosity of the matrix material 312, for example, by increasing the temperature.

As indicated symbolically on the basis of the bulk vessel 316 in FIG. 3, in a next method step, a magnetic component 318 is admixed with the matrix material 310 by said magnetic component being added to the matrix material 310 with stirring, for example.

The magnetic component 318 is preferably present in powder form. What have proved to be particularly suitable are iron powders or nickel powders or other ferromagnetic materials or mixtures thereof having a small particle size, preferably having particle sizes of below 400 micrometers, preferably less than 90 micrometers or having an average diameter of 60 micrometers.

A mixture that was used for producing a magnetic filling material 118 is specified by way of example below:

### Example 1

In a first exemplary embodiment, an epoxy resin of the type octite Hysol 9496 from Henkel AG & Co. KGaA in Düsseldorf, Germany was used as matrix material. This matrix material comprises an epoxy resin having a viscosity of more than 10 000 mPas as first component and an amine having a viscosity of above 200 mPas as second component. Six % by volume of the first component was admixed with approximately one % by volume of the second component (curing agent). The mixed matrix material has a viscosity of approximately 2 600 mPas.

In order to produce the magnetic filling material 118, in a first example, iron powder as magnetic component 318 was admixed with the abovementioned first component (epoxy component) of the matrix material 310. Iron powder of the type FE006010 from Goodfellow GmbH in Friedberg, Germany was used for this purpose. This iron powder has a purity of at least 99.0% and also a maximum particle size of 450 micrometers. This iron powder as magnetic component 318 was admixed with the first component of the matrix material 310 in a concentration of 15% by weight with stirring. Shortly before the processing of the magnetic filling material, finally, this mixture was admixed with the second component (curing agent) of the matrix material, and the magnetic filling material produced in this way was processed.

The filling material 118 produced in this way was used in various magnetic shields 110. In this case, it was possible to detect a significantly reduced reluctance at the transition locations between individual shielding elements 112, which proved the functionality of the proposed mixture.

### Example 2

Substantially the mixture described in example 1 was used in a second exemplary embodiment. Instead of iron powder of the type FE006010, however, iron powder of the type FE006020, likewise from Goodfellow GmbH in Friedberg, Germany, was used in this exemplary embodiment. This iron powder has a maximum particle size of 60 micrometers and a purity of likewise at least 99.0%. The magnetic filling material 118 was otherwise produced substantially analogously to example 1.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

### PARTS REFERENCE NUMERALS

110 Magnetic shield
112 Shielding element
114 Butt joint
116 Connecting region
118 Magnetic filling material
120 Syringe
210 Biomagnetic measurement system
212 Patient chamber
214 Interior space
216 Measurement container
218 Antichamber
220 Driving and evaluation electronics
222 Magnetic sensor system
224 Dewar vessel
226 Suspension device
228 Patient
230 Patient couch
310 Matrix material
312 Mixing vessel
314 Stirrer
316 Bulk vessel
318 Magnetic component

What is claimed is:

1. A magnetic filling material for bridging interspaces in magnetic shields and for shielding static or low-frequency magnetic fields, comprising:
at least one matrix material; and
at least one magnetic component embedded in the at least one matrix material, wherein the at least one magnetic component has magnetically shielding properties and comprises a material having a permeability of at least 100.

2. The magnetic filling material of claim 1, wherein the at least one matrix material has substantially diamagnetic properties.

3. The magnetic filling material of claim 1, wherein the at least one matrix material has a deformable state and a cured state, wherein the at least one matrix material is liquid or has at least partly plastic and/or elastic properties in the deformable state, and wherein the at least one matrix material is substantially rigid in the cured state.

4. The magnetic filling material of claim 1, wherein the at least one matrix material comprises an adhesive.

5. The magnetic filling material of claim 1, wherein the at least one matrix material comprises at a resin, a multi-component adhesive or at least one component of a multi-component adhesive.

6. The magnetic filling material of claim 1, wherein the at least one magnetic component comprises at least one ferromagnetic material having a permeability of at least 300.

7. The magnetic filling material of claim 1, wherein the at least one magnetic component comprises at least one ferromagnetic material having a permeability of at least 1000.

8. The magnetic filling material of claim 1, wherein the at least one magnetic component comprises a metallic material.

9. The magnetic filling material of claim 1, wherein the at least one magnetic component comprises at least one of the following materials: iron; nickel; an alloy comprising the elements iron and nickel comprising a nickel fraction of between 60% and 90%.

10. The magnetic filling material of claim 1, wherein the at least one magnetic component comprises an alloy comprising the elements iron and nickel and having a nickel fraction of between 75% and 80%.

11. The magnetic filling material of claim 1, wherein the at least one magnetic component comprises a powder or particles.

12. The magnetic filling material of claim 11, wherein the at least one magnetic component comprises particles having an average particle size of less than 500 micrometers.

13. The magnetic filling material of claim 12, wherein the particles have an average particle size of less than 90 micrometers.

14. The magnetic filling material of claim 12, wherein the particles have an average particle size of about 60 micrometers.

15. The magnetic filling material of claim 1, wherein the at least one magnetic component comprises a mass fraction of 5 to 50 weight percent of the magnetic filling material.

16. The magnetic filling material of claim 15, wherein the at least one magnetic component comprises a mass fraction of about 15 weight percent of the magnetic filling material.

17. The magnetic filling material of claim 1, wherein the at least one magnetic component comprises a volume fraction of 1 to 30 volume percent of the magnetic filling material.

18. A magnetic shield for shielding static or low-frequency magnetic fields, comprising:
a shielding element having magnetically shielding properties and having an opening; and
a magnetic filling material disposed in the opening, the magnetic filling material comprising a matrix material having a magnetic component embedded therein, wherein the magnetic component has magnetically shielding properties and is configured for reinforcing the magnetic shield.

19. The magnetic shield of claim 18, wherein the shielding element comprises first and second shielding elements and the opening comprises a connecting region at which the shielding elements are connected to one another, wherein the magnetic filling material is disposed in the connecting region and reinforces the magnetic shield.

20. The magnetic shield of claim 18, wherein the opening comprises at least one of the following elements: a leadthrough; a joining location; a seam location; and a butt joint.

21. The magnetic shield of claim 18, wherein the shielding elements comprise at least one of the following materials: iron; nickel; and an alloy comprising the elements iron and nickel and having a nickel fraction of between 60% and 90%.

22. The magnetic shield of claim 21, wherein the shielding elements comprise an alloy comprising the elements iron and nickel and having a nickel fraction of between 75% and 80%.

23. The magnetic shield of claim 18, wherein the shielding elements are configured substantially as plate-type elements.

24. The magnetic shield of claim 18, wherein the shielding elements have a permeability of at least 100.

25. The magnetic shield of claim 18, wherein the magnetic shield has a substantially parallelepipedal form.

26. The magnetic shield of claim 18, wherein the magnetic shield encloses an accessible interior space of at least 1 m³.

27. A biomagnetic measurement system for use in magnetocardiology or magnetoneurology, comprising:
a magnetic sensor system for detecting at least one magnetic field; and
a magnetic shield as claimed in claim 18.

28. A method for producing a magnetic shield, comprising:
connecting at least two shielding elements to one another and thereby forming a connecting region in an area where the two shielding elements are connected; and
introducing magnetic filling material into the connecting region, the magnetic filling material comprising a matrix material with a magnetic component embedded therein, the magnetic component having magnetically shielding properties.

29. The method of claim 28, further comprising introducing the magnetic filling material into the connecting region in a deformable state and then allowing the magnetic filling material to cure.

30. The method of claim 28, further comprising connecting the shielding elements to one another by a force-locking connection, a positively locking connection or a cohesive connection.

31. The method of claim 28, further comprising connecting the shielding elements to one another by a plug connection.

32. The method of claim 28, further comprising connecting the shielding elements to one another by welding.

33. The method of claim 28, further comprising mixing the magnetic component into the matrix material.
